SEQUENCE LISTING (4 sequences)

SEQ ID NO: 1 - protein, 516 aa, Synthetic construct
MSAVWSLGAGLLLLLLWVRHRGLEAVLVHHRWIFVCFFLMPLSILFDVYYQLRAWAVRRMHSAPRLHGQRVRHIQEQVREWKEEGGRRYMCTGRPGWLTVSLRVGKYKKTHKNIMINLMDVLEVDSERQVVRVEPLVTMGQLTAYLNPMGWTIPVVPELDDLTVGGLIMGTGIESSSHIYGLFQHTCMAYELVLADGSLVRCSPTENSDLFYAVPWSCGTLGFLVAAEIKMIPAKKYIRLHYEPVRGLRSICEKFTEESKNKENSFVEGLVYSLEEAVIMTGVLTDEAEPSKINRIGNYYKPWFFKHVEKYLKANKTGIEYIPSRHYYHRHTRSIFWELQDIIPFGNNPVFRYLFGWMVPPKISLLKLTQGEAIRKLYEQHHVVQDMLVPMKSLEKSIQTFHVDLNVYPLWLCPFLLPNNPGMVHPKGDETELYVDIGAYGEPKTKQFEARASMRQMEKFVRSVHGFQMLYADCYMTREEFWDMFDGSLYHSLREQMNCKDAFPEVYDKICKAARH

SEQ ID NO: 2 - protein, 228 aa, Synthetic construct
MGSDSILKHPYFPRDLSIPHYKPNKVSIQELLGIFFSLVLIVFIITWIYSTKKKFSVGYRLLLCWFTICGFIHTILEGYFAFNHSTLAGGSSYLAEMWKEYGKGDSRYITSDTFTVCMEGITAVIDGPLCFLTVAAYLTNSPYRYVLQLIVSLCQLYGDTLYLLSEVKDNFIHSELYHPLHFWFYFLFLNSFWIIVPLIMICHAVSKIVTSQGTSDFRFNNRNQKKSR

SEQ ID NO: 3 - DNA, 27 bp, Synthetic construct
  source 1..27: mol_type = other DNA
caatttggatcttctacctcttcccaa

SEQ ID NO: 4 - DNA, 210 bp, Synthetic construct
  source 1..210: mol_type = other DNA
ttcggccttctcagagatagaaccttgaacaggcttgttatctggagtataagggatttgaccagacacgtacacaaaattgttggccttcatagcttgggagtaagaggcggcagcgggtggggccaacttggtgctgaccggggtcaatgttgttatacccctcctcaagactggagctgttctcaaaacggaatttcttaaaaacat

(19) United States
(12) Patent Application Publication
(10) Pub. No.: US 2024/0002897 A1
(43) Pub. Date: Jan. 4, 2024

(54) METHOD FOR INCREASING YIELD OF 7-DEHYDROCHOLESTEROL IN YEAST BY USING COMPARTMENTALIZATION

(71) Applicant: JIANGNAN UNIVERSITY, Wuxi (CN)

(72) Inventors: Xueqin LV, Wuxi (CN); Long LIU, Wuxi (CN); Jian CHEN, Wuxi (CN); Guocheng Du, Wuxi (CN); Jianghua Li, Wuxi (CN); Yanfeng LIU, Wuxi (CN); Xiang XIU, Wuxi (CN)

(21) Appl. No.: 18/313,195

(22) Filed: May 5, 2023

Related U.S. Application Data
(63) Continuation of application No. PCT/CN2022/073487, filed on Jan. 24, 2022.

(30) Foreign Application Priority Data
Feb. 5, 2021 (CN) 202110160871.X

(51) Int. Cl. C12P 33/02; C12N 15/81; C12N 9/02; C12N 9/90

(52) U.S. Cl. CPC C12P 33/02; C12N 15/81; C12N 9/001; C12N 9/90; C12Y 103/01072; C12Y 503/03005

(57) ABSTRACT

The invention provides a method for increasing the yield of 7-dehydrocholesterol in yeast by using compartmentalization. The method includes steps of: by taking yeast as a starting strain, expressing heterologous sterol delta 24-reductase and cholestenol delta-isomerase, wherein the yield of 7-DHC in Saccharomyces cerevisiae S288C is detected to be 10.15 mg/L. According to the method of the invention, partial enzymes in a 7-DHC synthesis path are positioned in compartments in Saccharomyces cerevisiae by using a peroxisome and a mitochondrial positioning tag, and a relatively independent 7-DHC synthesis path is formed. Meanwhile, the storage space of precursor substances needed by 7-DHC synthesis is increased, the feedback effect is reduced, the conversion efficiency between enzymes is improved in the same compartment, the loss of the acting substrate is reduced, and finally the yield of the 7-DHC is improved by 4 times and reaches 53.31 mg/L.

This application is a Continuation Application of PCT/CN2022/073487, filed on Jan. 24, 2022, which claims priority to Chinese Patent Application No. 202110160871.X, filed on Feb. 5, 2021, which is incorporated by reference for all purposes as if fully set forth herein.

A Sequence Listing XML file named “10015_0118.xml” created on Sep. 23, 2023, and having a size of 5,241 bytes, is filed concurrently with the specification. The sequence listing contained in the XML file is part of the specification and is herein incorporated by reference in its entirety.

### FIELD OF THE INVENTION

The invention relates to the technical field of metabolic engineering, and in particular, to a method for increasing the yield of 7-dehydrocholesterol in yeast by using compartmentalization.

### DESCRIPTION OF THE RELATED ART

7-dehydrocholesterol is a sterol with high added value. The 7-dehydrocholesterol (7-DHC) synthesized in the human body is directly converted into vitamin D3 after being irradiated by sunlight in the skin tissue. Vitamin D3 is not only a fat-soluble vitamin necessary for the growth and development of human skeletal muscles, but also reduces the risk of immune system disorders and various cancers, and also has a preventive effect on cardiovascular diseases. The lack of vitamin D has been recognized as a global health problem, and also increases the annual global demand for vitamin D3 or its immediate precursor 7-DHC. 7-DHC is an important precursor for the synthesis of sterol drugs such as androstenedione and 9α-hydroxyandrost-4-ene-3,17-dione. 7-DHC not only has important applications in medicine and clinical practice, but also has been widely used in display manufacturing and other aspects as an important raw material for the preparation of cholesteric liquid crystal materials.

Traditional 7-DHC preparation methods are chemical synthesis method. There are two mainstream chemical synthesis methods. The first method uses cholesterol acetate as a raw material to synthesize 7-DHC through bromination, elimination and hydrolysis reactions at the 7-position. The second method uses cholesteryl acetate as a raw material to synthesize 7-DHC through oxidation, hydrazone, elimination and hydrolysis. The chemical synthesis methods have the problems of selectivity of reaction substrates, high energy consumption, harsh reaction conditions and serious pollution, which are not conducive to sustainable development. Methods for preparing 7-DHC by microbial fermentation are green, environmentally friendly and sustainable.

The methods for preparing 7-DHC by microbial fermentation mainly focus on the screening and molecular transformation of bacterial strains. The screening of bacterial strains is time-consuming and laborious, and large-scale screening is required. Even if the bacterial strains producing 7-DHC are found, the initial product concentration is extremely low, which is not conducive to the transformation and industrial production in the later stage. Molecular transformation mainly focuses on the elimination of branching pathways and the supply of precursor substances, but it is very easy to cause the accumulation of toxic intermediate products, which is not conducive to the growth and production of strains. Examples include Chinese patent CN107075551B, U.S. patent US2007/0204059A1, and Chinese patent CN109154015A. In Chinese patent CN109154015A, a sterol mixture is produced in yeast. In order to achieve the effect of preferential production of 7-DHC, heterologous enzymes for inactivating ERGS and ERG6, reducing or eliminating ARE2 and ARE1, and expressing the activity of sterol delta 24-reductase are required. However, the inactivation of key genes in the ergosterol synthesis pathway will definitely affect the growth of bacteria and cause the accumulation of intermediate products in the cytoplasm of yeast cells and metabolism balance.

The existing technologies for producing 7-DHC mainly focus on enhancing the mevalonate pathway in the cytoplasm, knocking out ERGS and ERG6 in the ergosterol synthesis pathway, and realizing the accumulation of 7-DHC in Saccharomyces cerevisiae. However, the 7-DHC synthesis path in Saccharomyces cerevisiae is long, and the key enzymes in the pathway are distributed in different organelles, leading to prominent problems of low conversion efficiency of enzymes to enzymes in the whole synthesis process, diffusion loss of substrates before the reaction, accumulation of intermediate products that inhibits feedback of early synthesis pathways, etc. As a result, the yield of producing 7-DHC in Saccharomyces cerevisiae cannot be greatly increased.

### SUMMARY OF THE INVENTION

In order to solve the current problem of insufficient acetyl-CoA in the synthesis of 7-DHC in the yeast cytoplasm, the invention uses the acetyl-CoA in peroxisomes and mitochondria to position the enzymes in the 7-DHC synthesis pathway through peroxisome and mitochondrial positioning protein tags respectively in mitochondria and peroxisomes, so that yeast can not only synthesize important 7-DHC in the cytoplasm, but also synthesize 7-DHC in peroxisomes and mitochondria, thereby improving the conversion efficiency of substrates in the metabolic pathway, increasing the storage space of the final product and reduces the feedback inhibition in the metabolic pathway. Finally, the yield of 7-DHC in yeast is 52.31 mg/L through this method.

A first objective of the invention is to provide a method for increasing yield of 7-dehydrocholesterol in yeast by using compartmentalization, expressing heterologous sterol delta 24-reductase and cholestenol delta-isomerase in peroxisomes and mitochondria of a yeast host respectively, and use a peroxisome positioning tag and a mitochondrial positioning tag to localize the enzymes in the 7-dehydrocholesterol synthesis pathway for expression in peroxisomes and mitochondria, respectively.

Further, the amino acid sequence of the sterol delta 24-reductase is as shown in SEQ ID NO: 1.

Further, the amino acid sequence of the cholestenol delta-isomerase is as shown in SEQ ID NO: 2.

Further, the enzymes in the 7-dehydrocholesterol synthesis pathway are hydroxymethylglutaryl-CoA synthase ERG13, mevalonate kinase ERG12, hydroxymethylglutaryl-CoA A reductase HMG1, phosphomevalonate kinase ERG8, mevalonate (diphospho) decarboxylase MVD1, isopentenyl diphosphate δ-isomerase IDI1, diphosphate farnesyltransferase ERG9, squalene mono oxygenase ERG1, lanosterol synthase ERG7, sterol 14α-demethylase ERG11, delta 14-sterol reductase ERG24, methylsterol monooxygenase ERG25, sterol 4α-carboxylate 3-hydrogenase ERG26, 3-ketosteroid reductase ERG27, delta 7-sterol 5-desaturase ERG3, sterol delta 24-reductase DHCR24 and cholestenol delta-isomerase EBP.

Further, the NCBI number of hydroxymethylglutaryl-CoA synthase ERG13 is XM_033912304.1, the NCBI number of mevalonate kinase ERG12 is XM_033912620.1, the NCBI number of hydroxymethylglutaryl-CoA A reductase HMG1 is XM_033912352.1, the NCBI number of phosphomevalonate kinase ERG8 is NM_001182727.1, the NCBI number of mevalonate (diphospho) decarboxylase MVD1 is NM_001183220.1, the NCBI number of isopentenyl diphosphate isomerase IDI1 is NM_001183931.1, the NCBI number of diphosphate farnesyltransferase ERG9 is NM_001179321.1, the NCBI number of squalene mono oxygenase ERG1 is NM_001181304.1, the NCBI number of lanosterol synthase ERG7 is NM_001179202.2, the NCBI number of sterol 14α-demethylase ERG11 is NM_001179137.1, the NCBI number of delta 14-sterol reductase ERG24 is NM_001183118.1, the NCBI number of methylsterol monooxygenase ERG25 is NM_001181189.3, the NCBI number of sterol 4α-carboxylate 3-hydrogenase ERG26 is NM_001180866.1, the NCBI number of 3-ketosteroid reductase ERG27 is NM_001181987.1, the NCBI number of delta 7-sterol 5-desaturase ERG3 is NM_001181943.1, the NCBI number of sterol delta 24-reductase DHCR24 is NM_001031288.1, and the NCBI number of cholestenol delta-isomerase EBP is KR709569.1.

Further, the peroxisome positioning tag is peroxisome positioning signal peptide PTS1, having a nucleotide sequence as shown in SEQ ID NO: 3.

Further, the mitochondrial positioning tag is a mitochondrial positioning signal peptide MMF1, having a nucleotide sequence as shown in SEQ ID NO: 4.

Further, the yeast host is Saccharomyces cerevisiae, Pichia pastoris or Candida tropicalis.

Further, the yeast host is Saccharomyces cerevisiae.

A second objective of the invention is to provide a yeast engineered bacterium. The yeast engineered bacterium expresses heterologous sterol delta 24-reductase and cholestenol delta-isomerase in peroxisomes and mitochondria of a yeast host respectively, and uses a peroxisome positioning tag and a mitochondrial positioning tag to express the enzymes in the 7-dehydrocholesterol synthesis pathway in peroxisomes and mitochondria respectively.

By virtue of the above solution, the present invention has at least the following advantages.

In the invention, by taking yeast as a starting strain, heterologous sterol delta 24-reductase and cholestenol delta-isomerase are expressed in peroxisomes and mitochondria at the same time. The yield of 7-DHC in Saccharomyces cerevisiae is detected to be 10.15 mg/L. According to the method disclosed by the invention, partial enzymes in a 7-DHC synthesis path are positioned in each compartmentalization in Saccharomyces cerevisiae by using an oxidase and a mitochondrial positioning tag, and a relatively independent 7-DHC synthesis path is formed. Meanwhile, the storage space of precursor substances needed by 7-DHC synthesis is increased, the feedback effect is reduced, the conversion efficiency between enzymes is improved in the same compartment, the loss of the acting substrate is reduced, and finally the yield of the 7-DHC is improved by 4 times and reaches 53.31 mg/L.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 is a schematic diagram of production of 7-DHC through yeast compartmentalization;

FIG. 2 is a liquid phase diagram of a 7-DHC standard product; and

FIG. 3 is a graph showing the liquid phase results of fermentation of Saccharomyces cerevisiae strain CDHC-17.

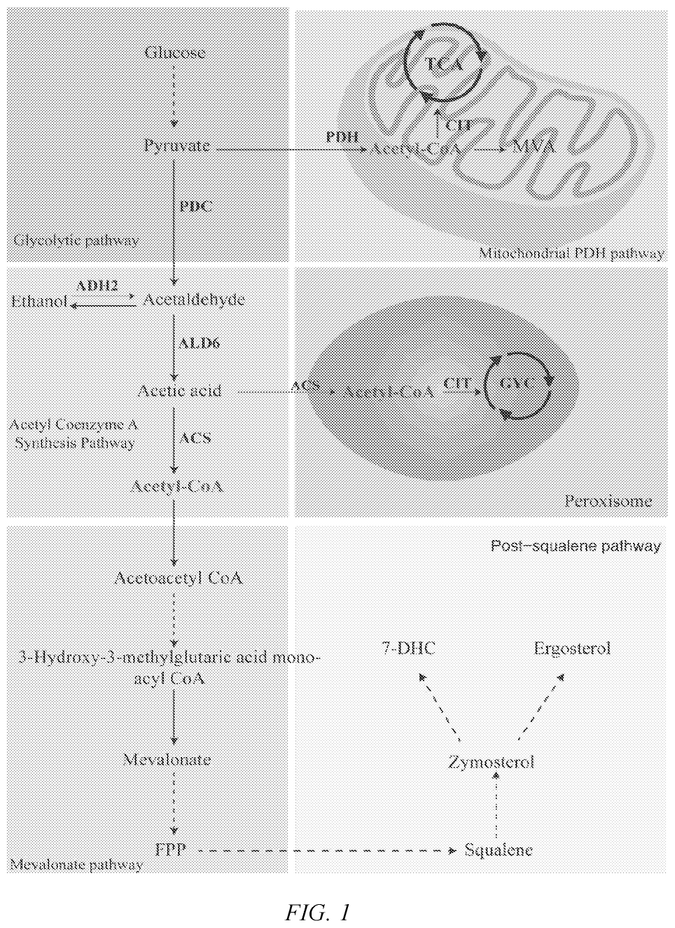

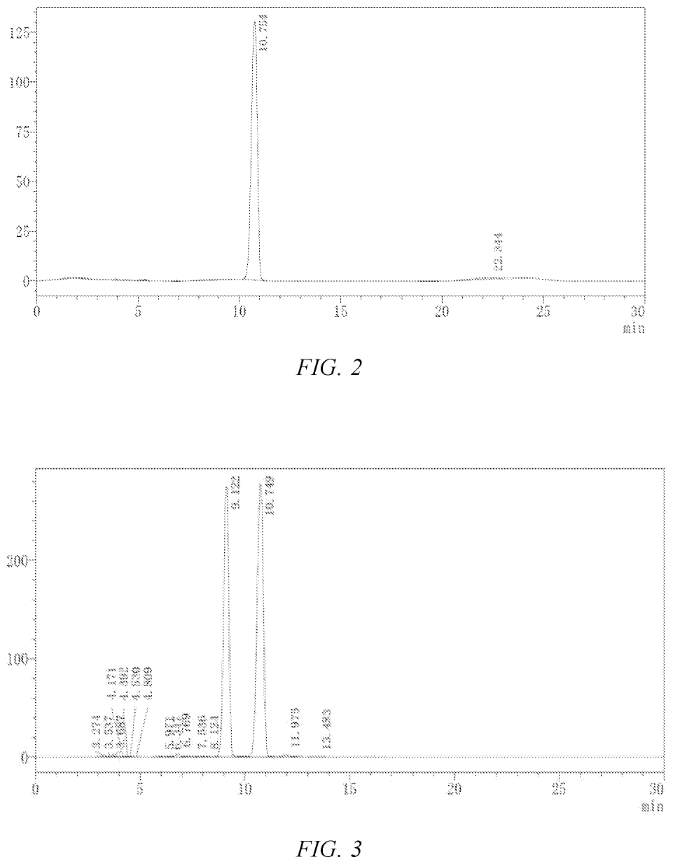

### DETAILED DESCRIPTION OF THE PREFERRED EMBODIMENTS

Involved detection methods were as follows.

7-DHC in Saccharomyces cerevisiae was tested by high performance liquid chromatography. A Saccharomyces cerevisiae liquid fermented for 60 hours was centrifuged, and resuspended in 10 ml of sterile water, followed by addition of 0.5 mm glass beads, and homogenization using a high-speed homogenizer for 10 minutes. The homogenized mixture was taken out, 1 g of ascorbic acid and 0.5 g of HBT were added, and mixed uniformly. 20 ml of absolute ethanol and 10 ml of 1.5 mol/L potassium hydroxide methanol solution were added in sequence for saponification in water at 80° C. for 2 hours. After the saponification was complete, the extracts (isopropanol:n-hexane=1:3) were ultrasonically agitated for 30 minutes. After the impurities in the lower layer were removed using a separatory funnel, the extract mixture was freeze-dried, and then redissolved using methanol or acetonitrile or a mixture of methanol and acetonitrile, filtered at 0.55 μm and analyzed by high performance liquid chromatography. The mobile phase was methanol and water, the detector was an ultraviolet detector, and the detection wavelength was 265 nm.

### Example 1: Construction of Saccharomyces cerevisiae CDHC-2

(a) Artificially synthesized gene fragment P_{TEF1}-DHCR24-T_{cyc1}-P_{GAP}-DIC-T_{ADH1} was amplified using Saccharomyces cerevisiae S228C genome as a template and primers 208F-F and 208F-R to obtain gene fragment 208-F, amplified using Saccharomyces cerevisiae S228C genome as a template and primers primer 208R-F and 208R-R to obtain gene fragment 208-R, and amplified using pMHyLp-trp plasmid as a template and primers LoxT-F and LoxT-R to obtain gene fragment LoxT.

(b) The four fragments P_{TEF1}-DHCR24-T_{cyc1}-P_{GAP}-DIC-T_{ADH1}, 208F-F, 208-R and LoxT obtained in step (a) were amplified by overlap extension PCR. After being verified to be correct by 1% agarose gel electrophoresis, the fragments were recovered by extraction to obtain a fusion gene fragment 208-F-LoxT-P_{TEF1}-DHCR24-T_{cyc1}-P_{GAP}-DIC-T_{ADH1}-208-R.

(c) The gene fragment obtained in step (b) was transformed into the competent strain of Saccharomyces cerevisiae S288C, coated on an SD Trp plate, cultured at for 3 days, and subjected to colony PCR verification using primers YZ-24-DIC-F and YZ-24-DIC-R to obtain strain CDHC-1.

(d) The strain CDHC-1 obtained in step (c) was made competent, and the plasmid PY26-Cre was transformed into Saccharomyces cerevisiae. After a single colony was grown on an SD Ura screening solid plate, the culture was inoculated into a YPD medium for 12 hours, and cultured on a YPD solid plate containing 5-FOA at for 3 days. Then the grown single colonies were transferred to a YPD solid plate and an SD Ura screening solid plate for comparison and verification. The single colony that grew normally on the YPD plate but cannot grow on the SD Ura screening plate was the correct genetically engineered bacterium, named CDHC-2.

Primer Sequence:

208F-F:
ccaggttaatgtgttctctgaaattcgc
208F-R:
gtaatcatggtcatagctgtttcctgttgtttagcggacgtgtgtatg
gt
208R-F:
cagcttttgttccctttagtgtttgacgatgtcagtgaatcccgg
208R-R:
cttgtagtccaccattgccatttttca
LoxT-F:
ccatacacacgtccgctaaacaacaggaaacagctatgaccatgatta
cg
LoxT-R:
acctttagtacgggtaattaacgataaaacgacggccagtgccaaggt
cg
YZ-24-DIC-F:
atggatttcgccaatcaaacccat
YZ-24-DIC-R:
caatgaaacagctggttccattcgaaactaagttctggtgttttaaaa
ctaa

### Example 2: Construction of Saccharomyces cerevisiae Mitochondrial Compartmentalization

(a) The Saccharomyces cerevisiae S228C genome was used as a template, and the mitochondrial positioning signal peptide MMF1 was selected. The gene fragment was amplified using primers ERG12-F and ERG12-MMF1-R to obtain gene fragment ERG12-MMF1. The gene fragment was amplified using primers ERG13-F1 and ERG13-MMF1-R1 to obtain gene fragment ERG13-MMF1. The gene fragment was amplified using primers GAL-F1 and GAL-R1 to obtain GAL1 promoter and GAL10 promoter gene fragments GAL1-10. The gene fragment was amplified using primers LoxT-GAL-F and LoxT-GAL-R to obtain gene fragment Lox-Trp. The gene fragment was amplified using primers GAL80F1-F and GAL80F1-R to obtain gene fragment GAL80F1. The gene fragment was amplified using primers GAL80R1-F and GAL80R1-R to obtain GAL80R1. The gene fragment was amplified using primers CYC1-S-F and CYC1-S-R to obtain CYC1. The gene fragment was amplified using primers ADH1-S-F and ADH1-S-R to obtain ADH1. The gene fragment was amplified using primers MMF1-F, MMF1-R, MMF2-F, and MMF2-R to obtain gene fragment MMF1-1 and MMF2-2.

(b) The CDHC-2 strain obtained in Example 1 was transformed into yeast competent cells. The 10 gene fragments obtained in step (a) were transformed into CDHC-2. Primers YZS-12-13-F and YZS-12-13-R were used for colony PCR verification.

(c) The correct single colony verified by colony PCR in step (d) was inoculated into SD trp liquid medium and cultured for 16 hours, and then streaked on a YPD solid plate containing 5-FOA at 30° C. for 3 days. Then the grown single colonies were transferred to a YPD solid plate and an SD his screening solid plate for comparison and verification. The single colony that grew normally on the YPD plate but cannot grow on the SD his screening plate was the correct genetically engineered bacterium, named CDHC-3.

(d) HMG1, ERGS, MVD1, IDI1, ERG9, ERG1, ERG7, ERG11, ERG24, ERG25, ERG26, ERG27, ERG3, DHCR24, and EBP genes were immobilized in mitochondria according to the above steps, and finally the strain CDHC-10 was obtained.

Primer Sequence:

ERG12-F1:
tcaaggagaaaaaactataatgtctcagaacgtttacattgtat
ERG12-MMF1-R:
aacggaatttcttaaaaacattatcttttcaatgacaatagaggaa
ERG13-F1:
tcttaaaaacatttttttaacatcgtaagatcttctaaat
ERG13-MMF1-R:
ttttgaaaattcaatataaatgaaactctcaactaaactttgttg
GAL-F1:
agttgagagtttcatttatattgaattttcaaaaattcttactttttt
tttgg
GAL-R1:
taaacgttctgagacattatagttttttctccttgacgttaaagt
LoxT-M-F:
cttattgaccacacctctaccggcaggaaacagctatgaccatgatta
cg
LoxT-M-R:
aatatcagagcatccataaaacgacggccagtgccaaggtcga
GAL80F2-F:
acaagttcgtacttttccaggataaatgc
GAL80F2-R:
ggctttaatttgcggccaccttgcattacaaattgtgagg
CYC1-M-F:
tgtaatgcaaggtggccgcaaattaaagccttcgagcgtc
CYC1-M-R:
atctctgagaaggccgaataatcatgtaattagttatgtcac
ADH1-M-F:
ctctgagaaggccgaagcgaatttcttatgatttatgatttttatt
ADH1-M-R:
tcatggtcatagctgtttcctgccggtagaggtgtggtcaataag
GAL80R2-F:
cactggccgtcgttttatggatgctctgatattacacaggttaat
GAL80R2-R:
accgggtgataggtttgctcaaccat
YZM-10-13-F:
ggaaaagctgcataaccactttaact
YZM-10-13-R:
ctgagaaagcaacctgacctacagg
MMF1-F:
tacatgattattcggccttctcagagatagaaccttgaac
MMF1-R:
atcttacgatgttaaaaaaatgtttttaagaaattccgtt
MMF2-F:
attgtcattgaaaagataatgtttttaagaaattccgttttgagaacag
MMF2-R:
aaatcataagaaattcgcttcggccttctcagagatagaa

### Example 3: Construction of Saccharomyces cerevisiae Peroxisome Compartmentalization

(a) The Saccharomyces cerevisiae S228C genome was used as a template, and the peroxisome positioning signal peptide PTS1 was selected. The gene fragment was amplified using primers ERG12-F and ERG12-PTS1-R to obtain gene fragment ERG12-PTS1. The gene fragment was amplified using primers ERG13-F and ERG13-PTS1-R to obtain gene fragment ERG13-PTS1. The gene fragment was amplified using primers GAL-F and GAL-R to obtain GAL1 promoter and GAL10 promoter gene fragments GAL1-10. The gene fragment was amplified using primers LoxT-GAL-F and LoxT-GAL-R to obtain gene fragment Lox-Trp. The gene fragment was amplified using primers GAL80F3-F and GAL80F3-R to obtain gene fragment GAL80F3. The gene fragment was amplified using primers GAL80R3-F and GAL80R3-R to obtain GAL80R3. The gene fragment was amplified using primers CYC1-F and CYC1-13-R to obtain CYC1. The gene fragment was amplified using primers ADH1-10-F and ADH1-R to obtain ADH1.

(b) The CDHC-10 strain obtained in Example 2 was transformed into yeast competent cells. The 8 gene fragments obtained in step (a) were transformed into CDHC-6. Primers YZ-10-13-F and YZ-10-13-R were used for colony PCR verification.

(c) The correct single colony verified by colony PCR in step (d) was inoculated into SD trp liquid medium and cultured for 16 hours, and then streaked on a YPD solid plate containing 5-FOA at 30° C. for 3 days. Then the grown single colonies were transferred to a YPD solid plate and an SD his screening solid plate for comparison and verification. The single colony that grew normally on the YPD plate but cannot grow on the SD his screening plate was the correct genetically engineered bacterium, named CDHC-7.

(d) HMG1, ERGS, MVD1, IDI1, ERG9, ERG1, ERG7, ERG11, ERG24, ERG25, ERG26, ERG27, ERG3, DHCR24, and EBP genes were immobilized in mitochondria according to the above steps, and finally the strain CDHC-17 was obtained.

Primer Sequence:

ERG12-F:
tcaaggagaaaaaactataatgtctcagaacgtttacattgtat
ERG12-PTS1-R:
ttgggaagaggtagaagatccaaattgtatcttttcaatgacaataga
ggaagca
ERG13-F:
acaatttggatcttctacctcttcccaattttttaacatcgtaag
ERG13-PTS1-R:
ttttgaaaattcaatataaatgaaactctcaactaaactttgttg
GAL-F:
agttgagagtttcatttatattgaattttcaaaaattcttactttttt
tttgg
GAL-R:
taaacgttctgagacattatagttttttctccttgacgttaaagt
LoxT-pts-F:
cttattgaccacacctctaccggcaggaaacagctatgaccatgatta
cg
LoxT-pts-R:
gcattactcaattttagactaaaacgacggccagtgccaaggtcg
GAL80F3-F:
ccaatgctaatccggtcactgccactgc
GAL80F3-R:
aggctttaatttgcggcctggcaatagaagtctcaatttt
CYC1-F:
gagacttctattgccaggccgcaaattaaagccttcgagc
CYC1-13-R:
gtagaagatccaaattgtaatcatgtaattagttatgtcacgctt
ADH1-10-F:
aatttggatcttctacctcttcccaagcgaatttcttatgattta
ADH1-R:
tcatggtcatagctgtttcctgccggtagaggtgtggtcaataag
GAL80R3-F:
gccgtcgttttagtctaaaattgagtaatgccactgcttttccca
GAL80R3-R:
atgtattgtaaaatatcgattgtgt
YZ-10-13-F:
ggaaaagctgcataaccactttaact
YZ-10-13-R:
ctgagaaagcaacctgacctacagg

### Example 4: Fermentation and Culture of Successfully Constructed Saccharomyces cerevisiae

A single colony of Saccharomyces cerevisiae CDHC-10 and a single colony of CDHC-17 were inoculated on a solid YPD plate in 2 ml of YPD medium. After culturing at 30° C. and 220 rpm for 16-20 hours, the colonies were inoculated at 1% of the inoculum size in a 250 mL round-bottomed shake flask containing 25 mL of YPD liquid culture, and cultured at 30° C. and 220 rpm for 60 hours. When fermented to 16 hours, ethanol was added to supplement the carbon source. After 108 hours of fermentation, the precipitate was collected by centrifugation, and the supernatant was removed. The precipitate was resuspended in 10 ml of sterile water, followed by addition of 0.5 mm glass beads, and homogenization using a high-speed homogenizer for 10 minutes. The homogenized mixture was taken out, 1 g of ascorbic acid and 0.5 g of HBT were added, and mixed uniformly. 20 ml of absolute ethanol and 10 ml of 1.5 mol/L potassium hydroxide methanol solution were added in sequence for saponification in water at 80° C. for 2 hours. After the saponification was complete, the extracts (isopropanol:n-hexane=1:3) were ultrasonically agitated for 30 minutes. After the impurities in the lower layer were removed using a separatory funnel, the extract mixture was freeze-dried, and then redissolved using methanol or acetonitrile or a mixture of methanol and acetonitrile, filtered at 0.55 μm and analyzed by high performance liquid chromatography. The mobile phase was methanol and water, the detector was an ultraviolet detector, and the detection wavelength was 265 nm.

After liquid phase analysis, the 7-DHC yield of the successfully constructed mitochondrial compartmentalized strain CDHC-10 was 29.35 mg/L. The 7-DHC yield of the peroxisome compartmentalized strain CDHC-17 successfully constructed on the basis of CDHC-10 was 53.31 mg/L.

While preferred embodiments of the present invention have been described above, the present invention is not limited thereto. It should be appreciated that some improvements and variations can be made by those skilled in the art without departing from the technical principles of the present invention, which are also contemplated to be within the scope of the present invention.

What is claimed is:

1. A method for increasing the yield of 7-dehydrocholesterol in yeast by using compartmentalization, comprising steps of: expressing heterologous sterol delta 24-reductase and cholestenol delta-isomerase in peroxisomes and mitochondria of a yeast host respectively, and using a peroxisome positioning tag and a mitochondrial positioning tag to localize the enzymes in the 7-dehydrocholesterol synthesis pathway for expression in peroxisomes and mitochondria, respectively.

2. The method according to claim 1, wherein an amino acid sequence of the sterol delta 24-reductase is as shown in SEQ ID NO: 1.

3. The method according to claim 1, wherein an amino acid sequence of the cholestenol delta-isomerase is as shown in SEQ ID NO: 2.

4. The method according to claim 1, wherein the enzymes in the 7-dehydrocholesterol synthesis pathway are hydroxymethylglutaryl-CoA synthase ERG13, mevalonate kinase ERG12, hydroxymethylglutaryl-CoA A reductase HMG1, phosphomevalonate kinase ERG8, mevalonate (diphospho) decarboxylase MVD1, isopentenyl diphosphate δ-isomerase IDI1, diphosphate farnesyltransferase ERGS, squalene mono oxygenase ERG1, lanosterol synthase ERG7, sterol 14α-demethylase ERG11, delta 14-sterol reductase ERG24, methylsterol monooxygenase ERG25, sterol 4α-carboxylate 3-dehydrogenase ERG26, 3-ketosteroid reductase ERG27, delta 7-sterol 5-desaturase ERG3, sterol delta 24-reductase DHCR24 and cholestenol delta-isomerase EBP.

5. The method according to claim 4, wherein the NCBI number of hydroxymethylglutaryl-CoA synthase ERG13 is XM_033912304.1, the NCBI number of mevalonate kinase ERG12 is XM_033912620.1, the NCBI number of hydroxymethylglutaryl-CoA A reductase HMG1 is XM_033912352.1, the NCBI number of phosphomevalonate kinase ERG8 is NM_001182727.1, the NCBI number of mevalonate (diphospho) decarboxylase MVD1 is NM_001183220.1, the NCBI number of isopentenyl diphosphate δ-isomerase IDI1 is NM_001183931.1, the NCBI number of diphosphate farnesyltransferase ERGS is NM_001179321.1, the NCBI number of squalene mono oxygenase ERG1 is NM_001181304.1, the NCBI number of lanosterol synthase ERG7 is NM_001179202.2, the NCBI number of sterol 14α-demethylase ERG11 is NM_001179137.1, the NCBI number of delta 14-sterol reductase ERG24 is NM_001183118.1, the NCBI number of methylsterol monooxygenase ERG25 is NM_001181189.3, the NCBI number of sterol 4α-carboxylate 3-hydrogenase ERG26 is NM_001180866.1, the NCBI number of 3-ketosteroid reductase ERG27 is NM_001181987.1, the NCBI number of delta 7-sterol ERG3 is NM_001181943.1, the NCBI number of sterol delta 24-reductase DHCR24 is NM_001031288.1, and the NCBI number of cholestenol delta-isomerase EBP is KR709569.1.

6. The method according to claim 1, wherein the peroxisome positioning tag is peroxisome positioning signal peptide PTS1, having a nucleotide sequence as shown in SEQ ID NO: 3.

7. The method according to claim 1, wherein the mitochondrial positioning tag is a mitochondrial positioning signal peptide MMF1, having a nucleotide sequence as shown in SEQ ID NO: 4.

8. The method according to claim 1, wherein the yeast host is Saccharomyces cerevisiae, Pichia pastoris or Candida tropicalis.

9. The method according to claim 8, wherein the yeast host is Saccharomyces cerevisiae S288C.

10. A yeast engineered bacterium, wherein the yeast engineered bacterium expresses heterologous sterol delta 24-reductase and cholestenol delta-isomerase in peroxisomes and mitochondria of a yeast host respectively, and uses a peroxisome positioning tag and a mitochondrial positioning tag to express the enzymes in the 7-dehydrocholesterol synthesis pathway in peroxisomes and mitochondria respectively.
